(19) United States
(12) Patent Application Publication
(10) Pub. No.: US 2011/0098536 A1
(43) Pub. Date: Apr. 28, 2011

(54) ILM Spatula with Malleable Blade

(76) Inventors: Borja F. Corcosteugi, Barcelona (ES); Ravi Nallakrishnan, Westmont, IL (US)

(21) Appl. No.: 12/265,246

(22) Filed: Nov. 5, 2008

(51) Int. Cl. A61B 1/32

(52) U.S. Cl. 600/206

(57) ABSTRACT

A surgical spatula has a blade with a tip formed with a portion with a first bendable portion beginning at the tip and a second, non-bendable portion extending from the terminus of the first blade portion toward the handle of the spatula. The blade is constructed in such a way as to allow the bending of the bendable portion by exerting very light pressure thereon making it possible for the surgeon to effect such bends during surgery without removing the instrument from the operating field.

### FIELD OF THE INVENTION

This invention relates generally to surgical instruments designed for use in eye surgery and, more particularly, to a blunt internal limiting membrane (ILM) pick or spatula having a malleable tip.

### BACKGROUND OF THE INVENTION

Certain ophthalmological surgical procedures require the peeling or delamination of the retinal ILM. A description of the various surgical techniques requiring delamination may be found in the second edition of Vitrious Microsurgery by Steve Charles, Williams and Wilkins, 1987, particularly at pages 120-121, 222, 163 and 164. Charles describes the use of a scissors to effect cuts in the ILM, reducing it to small enough pieces to be removed from the eye.

It is believed that the use of this “sharp scissors” technique carries with it an increased risk of injury to delicate tissues in the eye, for example, the nerve fiber layer. To minimize such risks, surgeons now use blunt dissection techniques, employing instruments such as the VRTIS™ ILM spatula sold by ASICO, LLC, of Westmont Ill. as its Model Nos. AE-2917 and AE-2921. Such a spatula includes a handle to which a shaft is mounted with the shaft terminating in a shaped tip having no sharp edges. The tip is typically contoured and slightly curved to enable manipulation of the spatula along the rear portion of the eye to perform a delamination of the ILM. The spatula tips on such instruments are formed from rigid stainless steel, meaning that the blade, while conveniently shaped for delamination of some portions of the ILM must be manipulated in order to bring it into a preferred position for delaminating a different portion of the ILM. In some cases, this manipulation involves rotating the handle of the spatula to, in effect, reverse the curve of the blade. It would be convenient to allow at least a portion of the tip to be bent in one direction or another while the instrument remains in the operating field without requiring undue manipulation of the handle.

Surgical instruments having deformable, bendable or malleable features are represented in the prior art.

U.S. Pat. No. 7,112,208 (Morris et al) teaches and describes a compact suture punch with malleable needle consisting of a hand held instrument used to grip the tissue and a trocar for forcing a malleable or bendable needle through the tissue to be sutured. In other words, the needle is straight as it enters the instrument but curves as it is performing the suturing operation.

U.S. Pat. No. 7,147,651 (Morrison et al) teaches and describes a stiff tipped suture whereby an otherwise entirely flexible suture is provided with a stiff or non-bendable tip to make it easier to thread the suture through a surgical instrument and, thereafter, through tissue.

U.S. Pat. No. 5,818,571 (Johnson) teaches and describes an apparatus for bending a surgical instrument. This instrument is designed to allow the tip of a phacoemulsification needle to be bent to a convenient angle prior to insertion into the eye but is intended for use with otherwise non-bendable tips.

### BRIEF DESCRIPTION OF THE INVENTION

An ILM spatula has a handle, a shaft extending from the handle and a tip formed on the distal end of the shaft. A portion of the tip is formed as a blade with a first non-bendable portion integral with and extending from the shaft and a second, bendable portion extending from the terminus of the first blade portion to the tip of the blade. The blade is constructed in such a way as to allow the bending of the bendable portion by exerting very light pressure thereon making it possible for the surgeon to effect such bends during surgery without removing the instrument from the operating field. The blade in an exemplary embodiment is somewhat flattened in shape and is bendable both upwards and downwards across the flattened portion of the blade. A degree of bend may also be effected from side to side across the blade as well. The blade can be bent to allow the tip to be inserted under the periphery of the ILM to lift or delaminate the ILM sufficiently to allow the use of other instruments to dissect the ILM for removal.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### DETAILED DESCRIPTION OF THE INVENTION

These and other aspects of the present invention will best be understood by reference to the accompanying drawings wherein:

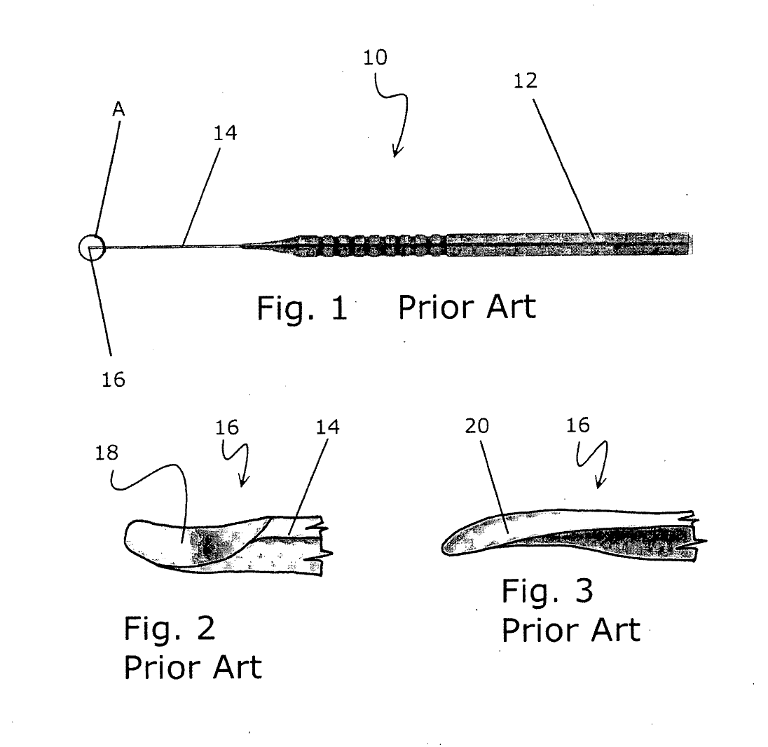

FIG. 1 is a lateral view of a prior art ILM spatula;

FIG. 2 is an enlarged view of the tip of the instrument shown in FIG. 1;

FIG. 3 is an enlarged view of the tip of FIG. 2 in a reversed position;

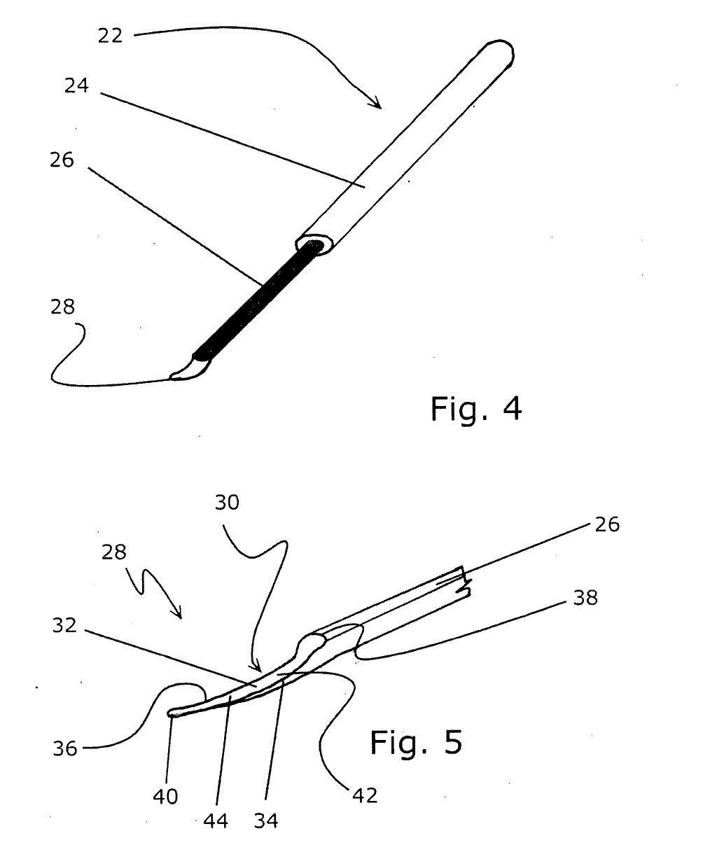

FIG. 4 is a schematic perspective view of an ILM spatula embodying principles of the present invention;

FIG. 5 is an enlarged view of the tip of the instrument shown in FIG. 4;

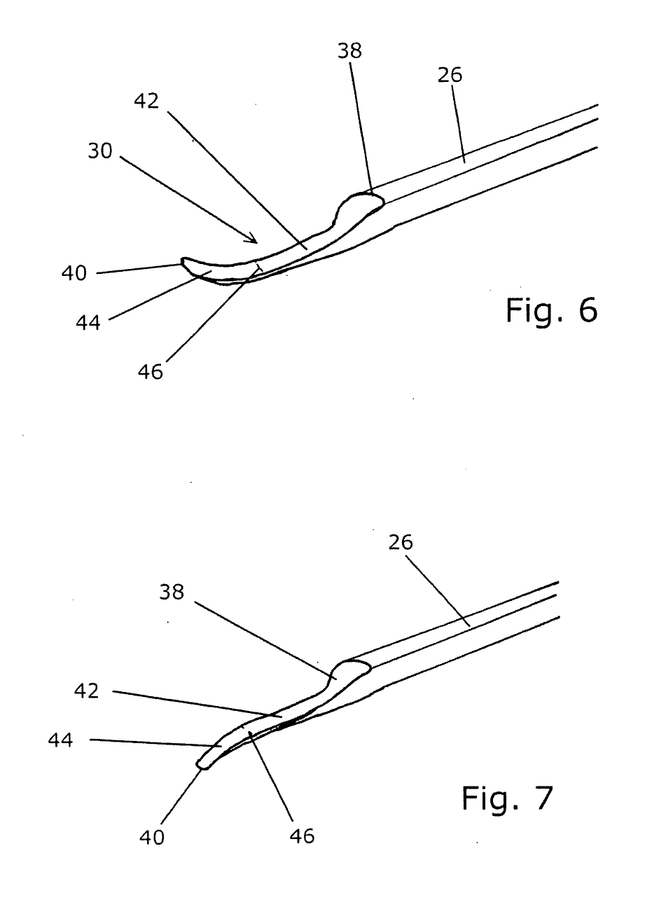

FIG. 6 is an enlarged view of FIG. 5 showing the blade bent in an upward direction;

FIG. 7 is a view of the tip shown in FIG. 5 with the blade bent in the downward direction;

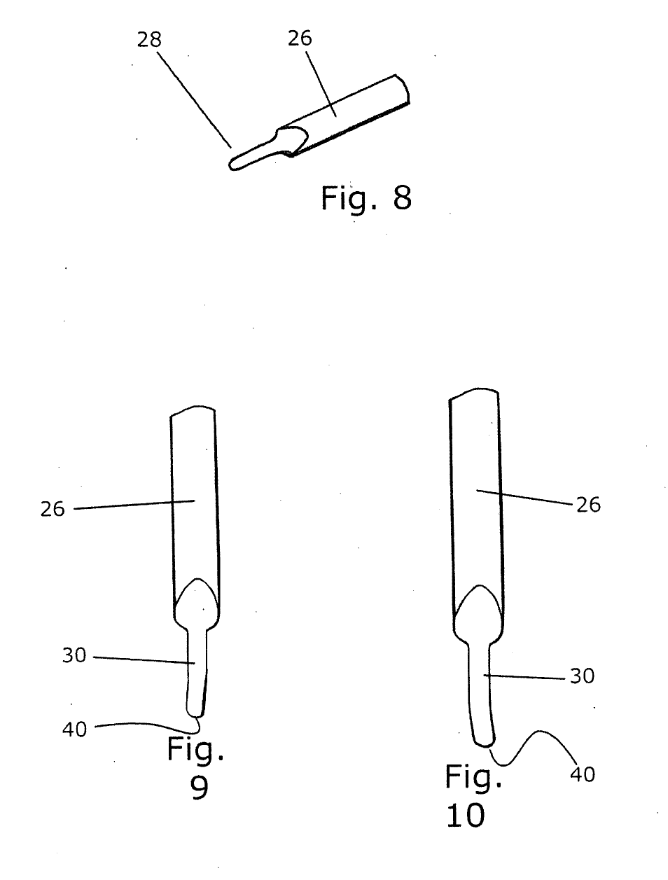

FIG. 8 is a partial perspective view of the blade of FIG. 4 in a “straight” position;

FIG. 9 is a view of the tip of FIG. 8 showing the blade bent laterally to the left; and

FIG. 10 is a view of the tip of FIG. 8 showing the blade bent laterally to the right.

### DETAILED DESCRIPTION OF THE DRAWINGS

Referring now to FIG. 1, the numeral 10 identifies a prior art ILM spatula having a handle 12, a shaft 14 attached at a proximal end to handle 12 and a blade 16. While the instrument is referred to herein as a “spatula” it is also referred to as a surgical pick. While the term “blade” is used herein, its context is a structure without sharp edges.

Referring now to FIG. 2, an enlarged view of blade 16 is shown as part of detail A of FIG. 1. Blade 16 has an upper surface or blade 18 which, in this view, is shown to have a slight curve to it.

Referring now to FIG. 3, blade 16 is shown inverted revealing a smooth, slightly curved bottom 20. As can be appreciated from these views, blade 16 is formed without a cutting edge.

In use, the position at which blade 16 contacts the ILM is changed by turning or rotating the handle 12 to move blade 16 to positions intermediate those shown in FIGS. 2 and 3.

Referring now to FIG. 4, the numeral 22 identifies a surgical spatula embodying certain of the principles of the present invention. Spatula 22 has a handle 24, and a shaft 26, attached at its proximal end to handle 24. At its distal end, shaft 26 terminates in a tip 28.

Referring now to FIG. 5, an enlarged view of tip 28 is shown. In this illustration, tip 28 comprises a blade 30 extending from shaft 26 and having an upper slightly curved surface 32, a left lateral wall 34 and a right lateral wall 36. As shown, blade 30 extends from shaft 26 at shoulder 38 and terminates at tip end 40.

Shaft 26 and blade 28 are typically formed from surgical stainless steel. It is a feature of the present invention that blade 30 is formed such that a first, stiff blade portion 42 extending from shoulder 38 toward tip end 40 is manufactured to be rigid and unbendable while a second, malleable blade portion 44 extending from stiff portion 42 to tip end 40 is manufactured to be malleable or bendable.

Typically, an instrument such as spatula 22 is manufactured from a single block of surgical stainless steel by use of a numerically computer-controlled (CNC) machining device. After machining, malleable portion 44 is treated, such as by annealing, to make it bendable.

The result of such a configuration is a spatula having a blade 30 that may, during surgery, be bent to a desired angle in order to more effectively use spatula 22 without removing it from the surgical operating field.

Referring now to FIG. 6, tip blade 30 is shown bent in an “upward” direction, that is, toward shaft 26 thereby angling malleable portion 44 upward. In this illustration, a bend axis 46, defined by the junction of stiff blade portion 42 and malleable blade portion 44 is formed intermediate tip end 40 and shoulder 38 and it is along this axis that blade 30 may be bent.

The position of bend axis 46 may be selected during the manufacturing process and may be different from instrument to instrument to give each instrument its own particular feel.

It is feature of the present invention to allow blade 30 to be bent about bend axis 46 in both an upward and downward direction, and the bending of tip blade 30 in a downward direction is illustrated in FIG. 7 with malleable portion 44 bent along bend axis 46 in a direction away from the top surface of shaft 26.

It is also intended that tip blade 30 have a degree of bend possible in a lateral direction with respect to shaft 26.

Referring now to FIG. 8, blade 30 is shown in a straight or unbent position. In FIG. 9, tip blade 30 is shown bent slightly in a side-to-side direction to the left as viewed, moving tip end 40 to the left. Similarly, as shown in FIG. 10, tip blade 30 is shown bent slightly to the right as viewed, moving tip end 40 to the right.

It is to be expected that the lateral bending of tip blade 30 may also be combined with the upward or downward bending in a relative twisting motion to produce a wide variety of selected positions for malleable blade portion 44 and tip end 40.

I claim:

1. A surgical instrument, said instrument comprising:
a handle having a proximal end;
a shaft extending from said proximal end;
a blade extending from said shaft,
said blade terminating at a blade tip,
said blade having a first portion beginning at said tip;
said blade having a second portion contiguous with and extending from said first blade portion toward said shaft;
said first and second blade portions meeting at a bend axis; and
said first blade portion being relatively deformable with respect to said second blade portion.

2. The apparatus as recited in claim 1 wherein said first blade portion is deformable with said second blade portion at said bend axis.

3. The apparatus as recited in claim 1 wherein said first blade portion is deformable about said bend axis in a direction toward said shaft.

4. The apparatus as recited in claim 1 wherein first blade portion is deformable at said bend axis in a side-to-side direction.

5. The apparatus as recited in claim 5 wherein said first blade portion is deformable at said bend axis in a twisting motion.
